(19) United States
(12) Patent Application Publication
(10) Pub. No.: US 2025/0017539 A1
(43) Pub. Date: Jan. 16, 2025

(54) Mobile Three-Dimensional (3d) Imaging Machine Based on X-Ray, and 3d Imaging Data Acquisition Method

(71) Applicant: E-COM Technology Ltd., Beijing (CN)

(72) Inventor: Lumin ZOU, Beijing (CN)

(21) Appl. No.: 18/769,444

(22) Filed: Jul. 11, 2024

(30) Foreign Application Priority Data
Jul. 11, 2023 (CN) 202310840752.8

(51) Int. Cl. A61B 6/00

(52) U.S. Cl. CPC A61B 6/4405; A61B 6/542; A61B 6/547

(57) ABSTRACT

The present disclosure belongs to the technical field of X-ray imaging, and particularly relates to a mobile three-dimensional (3D) imaging machine based on an X-ray, and a 3D imaging data acquisition method. The mobile 3D imaging machine based on an X-ray includes a machine body, and a limiting mechanism, an exposure imaging mechanism and a position parameter extraction mechanism which are mounted on the machine body. A relative position between the exposure imaging mechanism and an object to be detected is limited by arranging the limiting mechanism on the machine body, so that the exposure imaging mechanism is able to reach each preset relative position in sequence for exposure imaging, and then, relative position data corresponding to projection data is extracted through the position parameter extraction mechanism.

### CROSS-REFERENCE TO RELATED APPLICATIONS

This application claims priority to Chinese Patent Application No. 202310840752.8, filed on Jul. 11, 2023, which is incorporated herein by reference in its entirety.

### TECHNICAL FIELD

The present disclosure belongs to the technical field of X-ray imaging, and particularly relates to a mobile three-dimensional (3D) imaging machine based on an X-ray, and a 3D imaging data acquisition method.

### BACKGROUND OF THE INVENTION

Due to differences in density, thickness and other aspects among various tissues and organs in a human body, absorptions of X-rays irradiated thereon are different, resulting in changes in an intensity distribution of X-rays passing through the human body and carrying human information, so as to ultimately form X-ray information images. Based on this, people have successively developed a 2D film X-ray imaging technology, a computed radiography (CR), a digital radiography (DR), and a 3D computed tomography (CT). With a development of the technology and an increasing demand for applications, X-ray-based detection technologies such as the CR, the DR and the CT are also applied in fields such as non-destructive detection, industrial detection and safety inspection.

The CR and the DR are widely used due to their advantages such as low radiation and fast imaging, but their usage scenarios have many limitations. What is more prominent is that the CR and the DR may only perform 2D projection imaging, and a formed image is susceptible to interference from tissue structures at different thicknesses inside an object to be imaged, or from external substances, resulting in less information available for imaging. When the CR and the DR are used for anomaly judgment, the credibility is low. Although the CT is able to form a 3D structural image inside the object to be imaged, the CT requires many times of exposure around a circumferential direction of the object to be imaged, resulting in problems such as high radiation quantity, slow imaging speed, heavy device and high cost, thereby also limiting the promotion of the CT.

Therefore, how to form 3D imaging through fewer times of 2D X-ray imaging has become a future of the X-ray technology. With a development of computer technologies such as image processing, it has become possible to establish 3D images by a 3D image reconstruction technology using 2D imaging data.

### SUMMARY OF THE INVENTION

The inventor found that although some algorithms already support a use of multiple 2D images for 3D reconstruction, they not only require 2D image data, but also require an accurate distance between each of the multiple 2D images and an object to be imaged, a position of an incident X-ray on the object to be imaged, etc. This requires a mobile 3D imaging machine based on an X-ray to have a high-precision distance calculation device, a control device for strictly controlling angle and distance between an X-ray source, a detector and the object to be imaged, etc. Thus, the development of the 3D imaging technology is greatly limited.

In order to solve the above technical problems, the present disclosure aims to provide a mobile 3D imaging machine based on an X-ray, and a 3D imaging data acquisition method. A relative position between an exposure imaging mechanism and an object to be detected is limited by arranging a limiting mechanism on a machine body, so that the exposure imaging mechanism is able to reach each preset relative position in sequence for exposure imaging, and then, relative position data corresponding to projection data is extracted through a position parameter extraction mechanism. The imaging machine has advantages that a cooperation degree of the projection data and the relative position data is high, the contribution to 3D reconstruction is high, and accuracy requirements for a collection distance, an angle and the like are reduced. Meanwhile, it is ensured that high-quality data required for 3D reconstruction is able to be obtained, and the operation is simple and easy. Furthermore, the imaging machine is able to move to any position such as a ward or an examination room according to needs.

The technical solutions of the present disclosure are as follows:

One aspect of the present disclosure provides a mobile 3D imaging machine based on an X-ray, including: a machine body, and a limiting mechanism, an exposure imaging mechanism and a position parameter extraction mechanism which are mounted on the machine body, the limiting mechanism is configured to control a relative position between the exposure imaging mechanism and an object to be detected, so that the exposure imaging mechanism is able to reach different relative positions between the exposure imaging mechanism and the object to be detected in sequence; the exposure imaging mechanism is configured to respectively collect multiple projection data of the X-ray on the object to be detected at a plurality of relative positions; the position parameter extraction mechanism is configured to extract relative positions between the object to be detected and the exposure imaging mechanism corresponding to each of the multiple projection data, and/or relative position data between an X-ray source and a detector in the exposure imaging mechanism corresponding to each of the multiple projection data.

In some embodiments, the limiting mechanism includes at least one track in an arc shape mounted on the machine body, a motion block capable of moving along the at least one track, and a motion control module for controlling the motion block to move along the at least one track; and the X-ray source in the exposure imaging mechanism is correspondingly connected with the motion block.

In some embodiments, two tracks in the at least one track are arranged on the machine body at an interval side by side, and a rack is laid in each of the two tracks along a length direction of the each of the two tracks; the motion block includes a first mounting frame, connecting shafts penetrating through the first mounting frame, and a plurality of motion gears arranged on the connecting shafts, and each of the motion gears is respectively meshed with a rack on a corresponding track of the two tracks; and the X-ray source is correspondingly connected with the first mounting frame; or, two tracks in the at least one track are sliding rails, and the two tracks are arranged on the machine body at an interval side by side; the motion block includes a second mounting frame, connecting shafts penetrating through the second mounting frame, and a plurality of rollers arranged on the connecting shafts, and each of the plurality of rollers is respectively arranged in a corresponding sliding rail of the sliding rails; and the X-ray source is correspondingly connected with the second mounting frame; or, two tracks in the at least one track are limiting sliding rods, and the two limiting sliding rods are arranged on the machine body at an interval side by side; the motion block includes a plurality of sliding blocks sleeved on the limiting sliding rods respectively, and a third mounting frame for connecting the sliding blocks together; and the X-ray source is correspondingly connected with the plurality of sliding blocks.

In some embodiments, when the motion block includes motion gears or rollers, the motion control module includes a motor, a driving gear sleeved on an output shaft of the motor, and driven gears fixed on the connecting shafts, and the driving gear is correspondingly meshed with one driven gear of the driven gears; or, the motion control module includes a driving chain wheel, a driven chain wheel sleeved on one of the connecting shafts, a transmission chain for connecting the driving chain wheel and the driven chain wheel, and a rotating handle for driving the driving chain wheel to rotate; and the driving chain wheel is located at a center of a circle where the two tracks are located; or, the motion control module includes a driving synchronous wheel, a driven synchronous wheel sleeved on one of the connecting shafts, a synchronous belt for connecting the driven synchronous wheel and the driving synchronous wheel, and a rotating handle for driving the driving synchronous wheel to rotate; and the driving synchronous wheel is located at a center of a circle where the two tracks are located.

In some embodiments, when the two tracks are limiting sliding rods, the motion control module includes an electric telescopic rod disposed below a side of the limiting sliding rods, a first end of the electric telescopic rod is fixed on the machine body, and a second end of the electric telescopic rod is connected with the third mounting frame through a bearing.

In some embodiments, the position parameter extraction mechanism includes a revolution meter arranged on the connecting shafts, and is configured to extract a current position of the X-ray source according to a number of rotations of the connecting shafts; or, the position parameter extraction mechanism includes a plurality of optoelectronic couplers uniformly distributed along the length direction of the two tracks, and is configured to extract a current position of the X-ray source according to a position of the optoelectronic coupler that is blocked in the plurality of the optoelectronic couplers; or, the position parameter extraction mechanism includes a scale extraction camera, position scales arranged on an outer wall of one of the two tracks along the length direction of the two tracks, and a first pointer arranged on the exposure imaging mechanism and pointing to one of the position scales; or, the position parameter extraction mechanism includes a scale extraction camera, angle scales arranged on the driving chain wheel or the driving synchronous wheel, and a second pointer pointing to one of the angle scales.

In some embodiments, the machine body includes a mobile chassis, and an imaging control calculator, a high-voltage generator and a lifting frame which are arranged on the mobile chassis; the imaging control calculator is electrically connected with the high-voltage generator and the detector respectively; the high-voltage generator is electrically connected with the X-ray source; and the two tracks are mounted on the lifting frame.

In some embodiments, the lifting frame includes a rotating base arranged on the mobile chassis and a longitudinal sliding support rod arranged on the rotating base, and an end of the two tracks are mounted in the longitudinal sliding support rod.

In some embodiments, the mobile 3D imaging machine based on the X-ray further includes a wireless control calculator electrically connected with the imaging control calculator.

Another aspect of the present disclosure provides a 3D imaging data acquisition method, including: obtaining 3D imaging data by using any one of the above mobile 3D imaging machines based on the X-ray.

The present disclosure has the following beneficial effects:

1. For problems in an existing 3D imaging technology that it is required to have a high-precision distance calculation device, a control device and an angle calculation device for strictly controlling and calculating the angle among an X-ray source, a detector and an object to be imaged, etc, resulting in relatively high price and relatively cumbersome use of an existing 3D imaging device, and problems that a device is relatively large in volume and is not easy to move, or a device cannot move due to a need to collect accurate geometric information and other requirements, so that the 3D imaging technology is not suitable for widespread use and the development of the 3D imaging technology is greatly limited, the present disclosure provides a mobile 3D imaging machine based on an X-ray, which is able to move, to simply control a relative distance between an exposure imaging mechanism and an object to be detected, and to simply obtain geometric parameters. The imaging machine includes a machine body, and a limiting mechanism, an exposure imaging mechanism and a position parameter extraction mechanism which are mounted on the machine body. The limiting mechanism limits a motion of the exposure imaging mechanism to obtain multiple projection data at different relative positions, and then, relative position data is extracted through a simple position parameter extraction mechanism. Relevant parameters of a path for controlling X-rays to pass through the object is matched with relevant parameters of collection path, so that the cooperation degree of collected projection data and the relative position data is high, the contribution to 3D reconstruction is high, the relative position among the X-ray source, the detector and the object to be imaged is controlled simply and effectively, and accuracy requirements for a collection distance, an angle and the like are reduced, which helps to reduce the cost, improve the efficiency and maintain the imaging quality. Furthermore, the limiting mechanism, the exposure imaging mechanism and the like are able to be driven by the machine body to move to any position such as an examination room or a ward, and are able to be simply adjusted after movement to collect 2D projection data and relative position data required for reconstructing a high-quality 3D image. The application of this system for 3D imaging has advantages of less exposure times, low radiation, more imaging information, fast imaging speed, low price, high scalability, etc.

2. The limiting mechanism used in the present disclosure includes at least one track in an arc shape mounted on the machine body, a motion block capable of moving along the at least one track, and a motion control module for controlling the motion block to move along the at least one track; and the X-ray source in the exposure imaging mechanism is correspondingly connected with the motion block. When the motion block moves in the at least one track, the X-ray source is driven to move, and the characteristic of stable motion of the motion block on the at least one track is used for the exposure imaging mechanism, thereby ensuring the stability of the exposure imaging mechanism, effectively ensuring an accuracy of control for the exposure imaging mechanism, and also reducing motion artifacts. The at least one track is designed to be arc-shaped, which is favorable for limiting a distance between the X-ray source and the detector. When a center of a circle where the arc is located is at a certain point in an imaging region of the detector and a center of an X-ray beam also passes through the certain point, during a movement of the X-ray source on the at least one track, a beam center thereof is always located at the certain point, and an imaging distance remains unchanged. Therefore, when relative position parameters are collected, distance parameters do not need to be collected.

3. The design of the present disclosure includes a mobile chassis, and an imaging control calculator, a high-voltage generator and a lifting frame which are arranged on the mobile chassis; the imaging control calculator is electrically connected with the high-voltage generator; the high-voltage generator is electrically connected with the X-ray source; and two tracks are mounted on the lifting frame. Both the high-voltage generator and the imaging control calculator are arranged on the mobile chassis, and the mobile chassis drives each device to move, thereby avoiding a limitation of the imaging control calculator on the movement of the X-ray source, and ensuring a free movement of the mobile 3D imaging machine.

4. In the design of the present disclosure, the lifting frame includes a rotating base arranged on the mobile chassis and a longitudinal sliding support rod arranged on the rotating base, and an end of the track is mounted in the longitudinal sliding support rod. A direction of the longitudinal sliding support rod is able to be adjusted through the rotating base, and a height of the at least one track is able to be adjusted by adjusting the position of the at least one track on the longitudinal sliding support rod. Therefore, a direction of the at least one track on the machine body and a height of the at least one track are able to be adjusted through the lifting frame to adapt to the position of the object to be detected, thereby increasing the applicability of the design.

### BRIEF DESCRIPTION OF DRAWINGS

The accompanying drawings described here are intended to provide a further understanding of the present disclosure and constitute a part of the present application, and do not constitute a limitation on the present disclosure.

FIG. 1 shows a schematic structural view of Embodiment 1 of a mobile 3D imaging machine based on an X-ray in the present disclosure (a front view, namely a view obtained by cutting a part of a track and a part of a longitudinal sliding support rod along the dotted line in the figure);

FIG. 2 shows a schematic structural view of Embodiment 1 of a mobile 3D imaging machine based on an X-ray in the present disclosure (a top view, namely a view obtained by cutting upper parts of tracks along the dotted line in the figure);

FIG. 3 is a partial enlarged view of a part A in FIG. 2;

FIG. 4 shows a schematic structural view of a motion block in Embodiment 1 of a mobile 3D imaging machine based on an X-ray in the present disclosure;

FIG. 5 shows a schematic structural view of connection between a motion control module and gears in Embodiment 1 of a mobile 3D imaging machine based on an X-ray in the present disclosure (a cross-sectional view obtained by cutting a part of a first mounting frame along the dotted line in the figure);

FIG. 6 shows a schematic structural view of Embodiment 2 of a mobile 3D imaging machine based on an X-ray in the present disclosure (a front view, namely a view obtained by cutting a part of a track and a part of a longitudinal sliding support rod along the dotted line in the figure);

FIG. 7 shows a schematic structural view of Embodiment 2 of a mobile 3D imaging machine based on an X-ray in the present disclosure (a top view, namely a view obtained by cutting upper parts of tracks along the dotted line in the figure);

FIG. 8 shows a schematic structural view of a motion block in Embodiment 2 of a mobile 3D imaging machine based on an X-ray in the present disclosure;

FIG. 9 shows a schematic structural view of Embodiment 3 of a mobile 3D imaging machine based on an X-ray in the present disclosure (front view);

FIG. 10 shows a schematic structural view of a motion control module in Embodiment 3 of a mobile 3D imaging machine based on an X-ray in the present disclosure (a top view, namely a partial schematic view obtained by cutting upper parts of tracks along the dotted line in the figure);

FIG. 11 shows a schematic structural view of a position parameter extraction mechanism in Embodiment 4 of a mobile 3D imaging machine based on an X-ray in the present disclosure;

FIG. 12 shows a schematic structural view of a position parameter extraction mechanism in Embodiment 5 of a mobile 3D imaging machine based on an X-ray in the present disclosure;

FIG. 13 is a partial enlarged view of a part B in FIG. 12;

FIG. 14 shows a schematic structural view of Embodiment 6 of a mobile 3D imaging machine based on an X-ray in the present disclosure (front view); and

FIG. 15 shows a schematic structural view of Embodiment 6 of a mobile 3D imaging machine based on an X-ray in the present disclosure (top view).

### DETAILED DESCRIPTION OF THE INVENTION

In order to make the objectives, technical solutions and advantages of the present disclosure more clear, the present disclosure will be further described in detail below with reference to implementations and accompanying drawings. Here, the illustrative implementations and descriptions of the present disclosure are used for explaining the present disclosure, but are not intended to limit the present disclosure.

Here, it should also be noted that in order to avoid blurring the present disclosure due to unnecessary details, only the structures and/or processing steps closely related to the solutions according to the present disclosure are shown in the accompanying drawings, and other details that are not closely related to the present disclosure are omitted.

It should be emphasized that the term “include/comprise” when used herein refers to the existence of features, elements, steps or components, but does not exclude the existence or attachment of one or more other features, elements, steps or components. The descriptions of positions such as up, down, horizontal and vertical in the present application are only descriptions that match the accompanying drawings in embodiments, or descriptions of positions that people are accustomed to using, and using states, positions and the like are not limited.

Here, it should also be noted that in the case of no conflict, embodiments in the present application and features in the embodiments may be combined with each other.

As used herein, the phrase “reconstructed image” is not intended to exclude the implementation solution of the present disclosure that generate data representing images rather than visual images. Therefore, as used herein, the term “image” broadly refers to both visual images and data representing visual images.

According to a mobile 3D imaging machine based on an X-ray provided in the present disclosure, a limiting mechanism and a position parameter collection mechanism are combined with an exposure imaging mechanism in traditional 2D X-ray imaging. The limiting mechanism is arranged on a machine body, and a relative position between the exposure imaging mechanism and an object to be detected is limited by the limiting mechanism. When the imaging machine moves to a position for imaging, the limiting mechanism limits the exposure imaging mechanism to reach each relative position in sequence to obtain corresponding projection data and relative position data. 3D reconstruction is able to be performed according to the obtained data. The imaging machine has characteristics of simple structure, mobility, low accuracy requirements for relative position parameter extraction devices, etc.

### Embodiment 1

FIG. 1 to FIG. 5 show schematic views of a mobile 3D imaging machine based on an X-ray provided in the present disclosure.

As shown in FIG. 1, a mobile 3D imaging machine based on an X-ray provided in this embodiment includes: a machine body 1, and a limiting mechanism 2, an exposure imaging mechanism and a position parameter extraction mechanism which are mounted on the machine body 1.
the limiting mechanism is configured to control a relative position between the exposure imaging mechanism and an object to be detected, so that the exposure imaging mechanism is able to reach different relative positions between the exposure imaging mechanism and the object to be detected in sequence.
the exposure imaging mechanism is configured to respectively collect multiple projection data of the X-ray on the object to be detected at a plurality of relative positions; and the position parameter extraction mechanism is configured to extract the relative position between the object to be detected (not shown in the figure) and the exposure imaging mechanism corresponding to each of the multiple projection data, and/or relative position data between an X-ray source 101 and a detector (not shown in the figure) in the exposure imaging mechanism corresponding to each of the multiple projection data.

In the present disclosure, the limiting mechanism is used for limiting the relative position between the exposure imaging mechanism and the object to be detected to limit a motion path of the exposure imaging mechanism, so that the exposure imaging mechanism is able to gradually move to each preset relative position along a preset trajectory. Then, an exposure is performed at each relative position to obtain each of the multiple projection data, and the position parameter extraction mechanism is combined with the information of the each preset relative position and collected information to together obtain relative position data corresponding to each of the multiple projection data, so that the reconstruction is able to be performed subsequently according to the projection data and the relative position data to obtain a 3D image. The system is able to be used for detection of human tissue structures and is also able to be used for detection of objects. Those skilled in the art are able to determine exposure parameters and the number and angle of 2D images required for reconstructing 3D images according to types, sizes and the like of actually collected objects. Before the system is used for collecting 2D data, when each relative position is determined, the number of required 2D images and corresponding relative positions are able to be preferentially selected according to position data such as imaging number and angle required for reconstructing a 3D image, and then, the relative position that the exposure imaging mechanism needs to reach is determined according to preferentially selected 2D images and the relative positions. By collecting 2D image data obtained by limited exposure and corresponding relative position data, 3D imaging data is able to be reconstructed.

As shown in FIG. 1 to FIG. 5, the limiting mechanism 2 in this embodiment includes at least one track 21, a motion block 22 capable of moving along the at least one track, and a motion control module for controlling the motion block to move along the at least one track; and the X-ray source 101 in the exposure imaging mechanism is correspondingly connected with the motion block.

In order to ensure that a distance between the exposure imaging mechanism and the object to be detected remains unchanged during a movement of the exposure imaging mechanism at each relative position, the at least one track in this embodiment is arc-shaped, and a center of a circle where the arc is located is on an upper surface of the detector. During a movement of the X-ray source driven by a first mounting frame, a distance between the X-ray source and the detector remains unchanged, and a focus of the X-ray source remains on the detector.

The arc-shaped design of the at least one track in this embodiment also helps to limit an imaging region of the exposure imaging mechanism, a center point of the X-ray source irradiating on the detector, etc, prevents a problem which is caused by a motion that X-rays emitted by the X-ray source cannot be accurately projected onto the detector or cannot effectively pass through the object to be detected, or only a part of the imaging region is located on the detector affecting 3D reconstruction.

As shown in FIG. 1 to FIG. 5, two tracks 21 in the at least one track in this embodiment are arranged on the machine body 1 at an interval side by side, and a rack 2101 is laid in each of the two tracks 21 along the length direction of the each of the two tracks 21; the motion block 22 includes a first mounting frame 2201, connecting shafts 2202 threaded on the first mounting frame 2201, and a plurality of motion gears 2203 arranged on the connecting shafts 2202, and each of the motion gears 2203 is respectively meshed with a rack 2101 on a corresponding track of the two tracks; and the X-ray source is correspondingly connected with the first mounting frame. In this embodiment, the X-ray source and the first mounting frame are welded by a welding extension frame; and two welding extension frames are connected with a bottom of the X-ray source, the two welding extension frames are respectively welded to front and rear walls of the first mounting frame, and an X-ray beam emission port is opposite to a gap between the front and rear walls of the first mounting frame, thereby preventing the limiting mechanism from blocking X-ray beams.

In this embodiment, the racks are meshed with the motion gears to limit a movement of the motion block in the two tracks, which not only limits a motion direction of the motion block, but also helps to limit a position of the X-ray source in the two tracks, so that the X-ray source accurately reaches each relative position and is firmly located in the relative position during exposure imaging to prevent relative shaking of the X-ray source during exposure imaging and then avoid motion artifacts. That is to say, the design effectively ensures the accuracy of the obtained 3D imaging data.

In order to ensure the stability of motion of the motion gears and the stability of the exposure imaging mechanism on the first mounting frame, in the mobile 3D imaging machine based on the X-ray provided in this embodiment as shown in FIG. 1 to FIG. 5, two connecting shafts 2202 arranged at an interval are penetrated on the first mounting frame 2201, and each of the connecting shafts 2202 is provided with a plurality of motion gears 2203; each of the motion gears 2203 on the connecting shafts is respectively meshed with the rack 2101 on the corresponding track; and the motion control module is connected with at least one of the connecting shafts.

In order to control a number of parts in the mobile 3D imaging machine based on the X-ray and increase an assembly convenience, in this embodiment, both ends of the two connecting shafts are respectively penetrated with one motion gear, and the motion gears at both ends of each of the connecting shafts are correspondingly meshed with the racks in the two tracks respectively. Of course, those skilled in the art may also increase the number of motion gears penetrated on the connecting shafts according to needs, so that one rack is able to be simultaneously meshed with the plurality of motion gears. At this time, increasing the number of motion gears is equivalent to increasing a width of the motion gears meshed with the racks, which is beneficial for maintaining the stability of motion of the motion gears on the racks. Of course, the increase in the number of motion gears also increases an assembly difficulty. In this embodiment, two tracks are provided, and the rack is arranged in each of the two tracks. The design of the two tracks is to ensure the stability of motion of the first mounting frame. Of course, in order to further improve the stability of motion, the number of tracks and racks may also be increased. The more tracks and racks there are, the more beneficial it is to maintain the stability of motion of the first mounting frame and the motion gears, but the more the number is, the greater the assembly difficulty is and the higher the cost is. Those skilled in the art may select the number of motion gears arranged on the connecting shafts according to actual needs, and may also select the number of tracks according to actual needs. Details are not elaborated here.

The motion control module in the present disclosure may be a mechanism for connecting a rotating handle on one connecting shaft, or other mechanisms for manual rotating transmission. The connecting shafts are manually operated to rotate forward and backward to drive the first mounting frame to move between both ends of the tracks. Of course, electric or pneumatic devices may also be configured to drive the connecting shafts to rotate. The following provides an electric motion control module used in this embodiment.

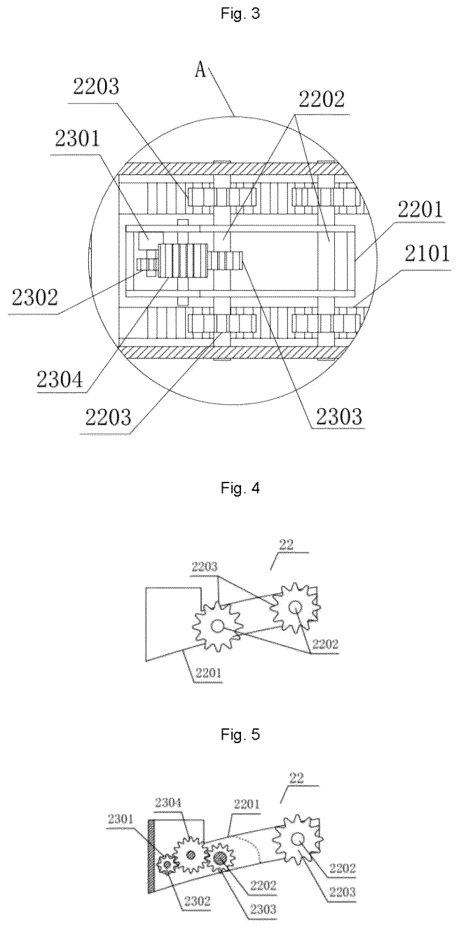

As shown in FIG. 3 to FIG. 5 and FIG. 7, in this embodiment, the motion control module includes a motor 2301, a driving gear 2302 sleeved on an output shaft of the motor 2301, and driven gears 2303 fixed on the connecting shafts, and the driving gear 2302 is correspondingly meshed with one of the driven gears 2303 through a transmission gear 2304. In this embodiment, the motor is connected with a reverse switch to control the motor to rotate forward or backward, thereby driving the connecting shafts to move forward or backward in the tracks.

As shown in FIG. 1 to FIG. 5, in this embodiment, the position parameter extraction mechanism includes a revolution meter (not shown in the figures) arranged on the connecting shafts 2202, and is configured to extract a current position of the exposure imaging mechanism according to the number of rotations of the connecting shafts. The motor 2301 drives the connecting shafts 2202 to rotate through a gear transmission mechanism, and then, the connecting shafts 2202 drive the motion gears 2203 to rotate, thereby completing the movement of the first mounting frame 2201 and the X-ray source 101 of the exposure imaging mechanism on the first mounting frame 2201 in the tracks 21, realizing a conversion of rotation into forward and backward movement, and strictly limiting a conversion relationship between the rotation and the forward and backward movement. Therefore, a distance of translational movement of the first mounting frame is able to be determined by counting a number of revolutions of the connecting shafts, a gear transmission ratio, etc, and then, the position of the exposure imaging mechanism during exposure is able to be directly determined according to the distance of translational movement of the first mounting frame. In this embodiment, the revolution meter has a small number of components and a simple structure and is easy to disassemble and assemble, the structure is hidden in the first mounting frame and thus is not easily damaged, and the output result is less susceptible to interference.

In some embodiments, the focus of the X-ray source is located on a geometric center of the detector. By counting the number of revolutions of the connecting shafts, an angle of the X-ray incident on the detector is able to be judged, thereby facilitating subsequent back projection transformation or other calculations to reconstruct a 3D image.

The above method provided in this embodiment is able to obtain a relative position between the X-ray source and the detector by calculating. Of course, a plurality of optoelectronic couplers uniformly distributed along the length direction of the tracks may also be used, thereby extracting the current position of the exposure imaging mechanism according to the position of the optoelectronic coupler that is blocked. The optoelectronic couplers are used as the position parameter extraction mechanism.

In order to further ensure a smooth motion of the first mounting frame and the exposure imaging mechanism, as shown in FIG. 1 to FIG. 3, in this embodiment, each of the tracks is provided with a connecting shaft penetration port 2102, and both ends of the connecting shafts 2201 are respectively arranged in the two connecting shaft penetration ports 2102. The connecting shafts penetrated from the connecting shaft threading ports 2102 may be configured to connect more motion gears and meshed with more racks, may also be configured to mount other types of motion driving mechanism, and may further be configured to determine the position of the exposure imaging mechanism and the like according to the position of the connecting shafts that are extended on the tracks. Those skilled in the art may match corresponding auxiliary structures or algorithms according to actually required functions etc. Details are not elaborated here.

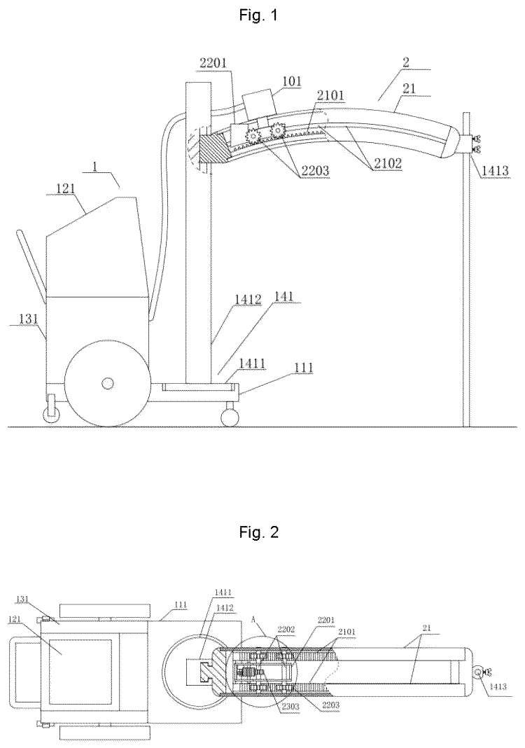

As shown in FIG. 1 and FIG. 2, the machine body 1 in this embodiment includes a mobile chassis 111, and an imaging control calculator 121, a high-voltage generator 131 and a lifting frame 141 which are arranged on the mobile chassis; the imaging control calculator 121 is electrically connected with the high-voltage generator and the detector respectively; the high-voltage generator is electrically connected with the X-ray source; and the two tracks are mounted on the lifting frame. Imaging control software is arranged in the imaging control calculator, and is configured to send an exposure control signal to the high-voltage generator and also configured to receive imaging data of the detector.

As shown in FIG. 1 and FIG. 2, the lifting frame 141 in this embodiment includes a rotating base 1411 arranged on the mobile chassis and a longitudinal sliding support rod 1412 arranged on the rotating base 1411, and an end of the two tracks are mounted in the longitudinal sliding support rod and may slide along a length direction of the longitudinal sliding support rod. In order to ensure the stability of the lifting frame, prevent the change in the center of a device caused by the position of movement of the X-ray source on the two tracks, and prevent the device from tilting, the design of connecting at least one longitudinal telescopic rod 1413 to an end of the track away from the longitudinal sliding support rod is used. During use, the bottom end of the longitudinal telescopic rod is supported on the ground or other firm objects. The longitudinal telescopic rod ensures that the two tracks will not shake up and down during the movement of the X-ray source on the two tracks, thereby ensuring the accuracy of imaging data.

This embodiment further includes a wireless control calculator electrically connected with the imaging control calculator so as to remotely control the exposure imaging mechanism for exposure, thereby reducing the radiation to operators.

### Embodiment 2

This embodiment is improved on the basis of Embodiment 1, and repeated parts are not elaborated here. The specific structures of the tracks and motion block in the limiting mechanism in this embodiment are different from those in Embodiment 1. The structures of the tracks and motion block in the limiting mechanism in this embodiment are described below.

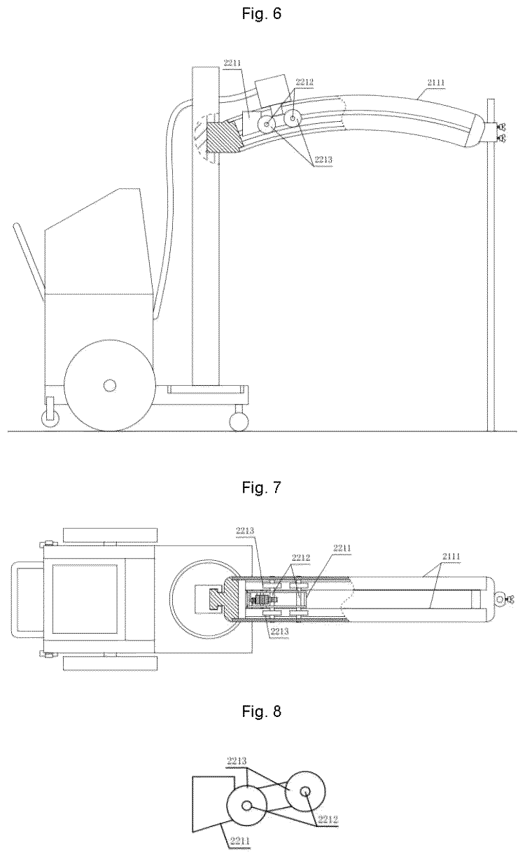

As shown in FIG. 6 to FIG. 8, in this embodiment, two tracks are sliding rails 2111, and the two tracks are arranged on a machine body at an interval side by side; the motion block includes a second mounting frame 2211, connecting shafts 2212 penetrated on the second mounting frame 2211, and a plurality of rollers 2213 arranged on the connecting shafts 2212, and each of the plurality of rollers 2213 is respectively arranged in the corresponding sliding rail of the sliding rails; and the X-ray source is correspondingly connected with the second mounting frame.

As shown in FIG. 6 to FIG. 8, in this embodiment, two connecting shafts 2212 arranged at an interval are penetrated on the second mounting frame, and each of the two connecting shafts is provided with the plurality of rollers 2213; each of the plurality of rollers 2213 on the connecting shafts 2212 is respectively arranged in the corresponding sliding rail; and the motion control module is connected with at least one of the two connecting shafts. In this embodiment, each of the two connecting shafts is respectively provided with two rollers 2213; and both ends of the two connecting shafts penetrate through the second mounting frame and then are respectively connected with one of the rollers.

In order to improve the stability of rolling motion between the rollers and the sliding rails and prevent the rollers from sliding on the sliding rails, elastic cushions are laid in the sliding rails to increase the friction force between the rollers and the two tracks. In this embodiment, the material of the elastic cushions is silicone. Of course, the material may also be nano glue with slight viscosity or rubber. The material may be selected by those skilled in the art according to actual needs. Of course, both the surface of the rollers and the surface of the two tracks may be provided with a frosted layer to increase the friction force between the rollers and the two track, thereby preventing sliding.

This embodiment uses the characteristic of the rollers moving in the two tracks and being able to stop at any position in the two tracks at any time during the movement of the rollers, which helps to set a relative position at any position and facilitates an arbitrary adjustment of the position, number and spacing between two adjacent relative positions for collected projection data, thereby improving the flexibility of this system.

### Embodiment 3

This embodiment is changed on the basis of Embodiment 1 or Embodiment 2, and repeated parts are not elaborated here. The motion control module in this embodiment is different from the motion control module in the above embodiment. The structure of the motion control module in this embodiment is briefly described below.

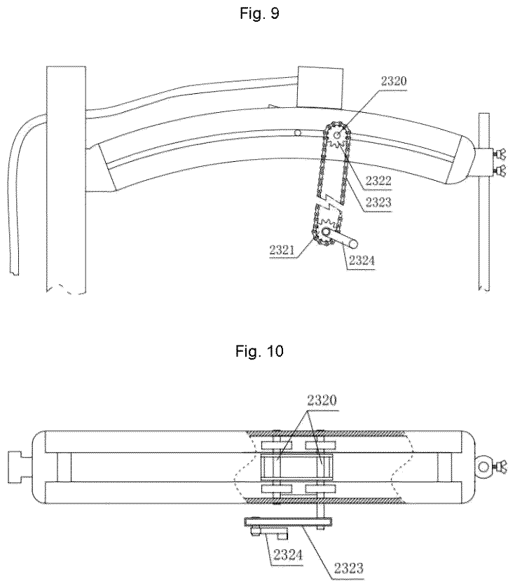

As shown in FIG. 9 and FIG. 10, in this embodiment, the motion control module includes a driving chain wheel 2321, a driven chain wheel 2322 sleeved on one of the connecting shafts 2320, a transmission chain 2323 for connecting the driving chain wheel 2321 and the driven chain wheel 2322, and a rotating handle 2324 for driving the driving chain wheel to rotate; and the driving chain wheel is located at a center of a circle where the two tracks 21 are located. The driving chain wheel is fixed on a stable bracket, base or detector mounting frame through a fixing device. One of the connecting shafts in the motion block penetrates through a side wall of one of the two tracks and is connected with the driven chain wheel, the rotating handle drives the driving chain wheel to rotate under the mechanical or manual action, and the driving chain wheel drives the driven chain wheel to rotate through the transmission chain. Because the one of the connecting shafts where the driven chain wheel is located is mobile and the driving chain wheel is located at the center of the circle where the two tracks are located, the rollers or motion gears on the connecting shafts are able to roll in the two tracks, and the motion direction of the motion block in the two tracks is able to be adjusted by adjusting the rotation direction of the driving chain wheel, thereby driving the exposure imaging mechanism to move to each relative position in sequence.

The motion control module provided in this embodiment has the advantages of high control accuracy, low inertia influence, fast response speed and simple direction control.

In this embodiment, the motion control module includes chain wheels and a chain. The chain and the chain wheels are usually made of metals having relatively large mass, therefore, it is not beneficial for controlling a weight of this system. In order to further reduce the weight of this system, synchronous wheels and a synchronous belt may also be used for transmission to drive the connecting shafts to rotate. At this time, the motion control module includes a driving synchronous wheel, a driven synchronous wheel sleeved on one of the connecting shafts, a synchronous belt for connecting the driven synchronous wheel and the driving synchronous wheel, and a rotating handle for driving the driving synchronous wheel to rotate; and the driving synchronous wheel is located at the center of the circle where the two tracks are located.

The synchronous belt and the synchronous wheel are in the prior art. The manner of driving the connecting shafts and the exposure imaging mechanism through the synchronous belt, the driving synchronous wheel, the driven synchronous wheel and the like is the same as the manner of driving the connecting shafts and the exposure imaging mechanism through the transmission chain, the driving chain wheel, the driven chain wheel and the like in this embodiment, so those skilled in the art are able to refer to chain and chain wheel mounting manners for design. Details are not elaborated here.

### Embodiment 4

This embodiment is improved on the basis of Embodiment 1, 2 or 3, and repeated parts are not elaborated here. The position parameter extraction mechanism in this embodiment is different from that in the above embodiment. The structure of the position parameter extraction mechanism in this embodiment is briefly described below.

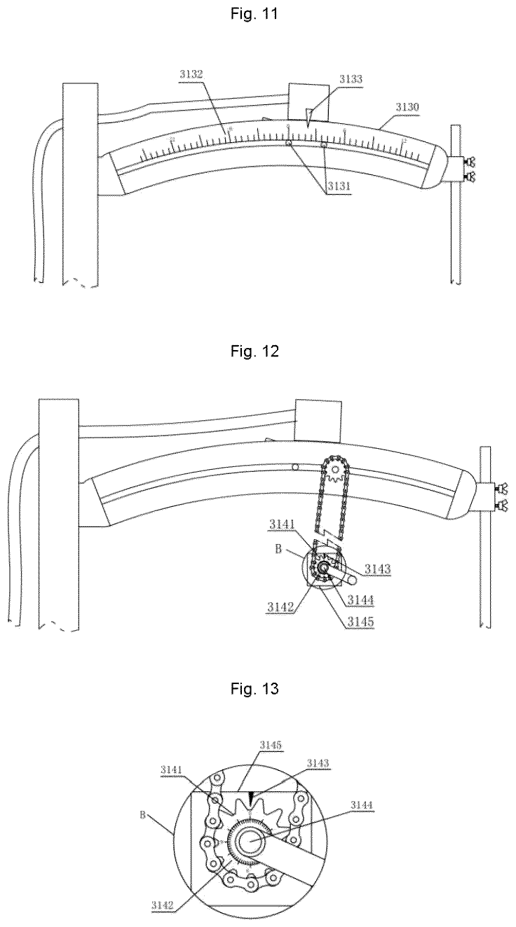

As shown in FIG. 11, in this embodiment, the position parameter extraction mechanism includes a scale extraction camera (not shown in the figure), position scales 3132 arranged on an outer wall of one of the two tracks along the length direction of the two tracks 3130, and a first pointer 3133 arranged on the X-ray source and pointing to the position scales.

In this embodiment, the first pointer is arranged on the exposure imaging mechanism. Of course, the first pointer 3133 may also be connected with the connecting shaft 3131. The position scales are attached to the outer wall of one of the two tracks. When the connecting shafts and the exposure imaging mechanism move along the two tracks, the first pointer is driven to move. A scale extraction camera which has a certain distance from the track which the position scales are attached to, a shooting direction facing the track and a shooting format capable of covering entire scales is configured to obtain a position scale pointed by the first pointer at each relative position or during each time of exposure, thereby accurately determining the relative position between the exposure imaging mechanism and the object to be detected, or between the X-ray source and the detector.

The position parameter extraction mechanism provided in this embodiment is able to directly read the movement distance or position of the exposure imaging mechanism, and a penetration path of the X-ray incident on the detector is able to be calculated according to the movement distance or position, thereby performing 3D reconstruction based on this and projection data at each relative position.

In some embodiments, the position scale refers to an angle at which the X-ray is irradiated into the object to be detected or the detector when the exposure imaging mechanism is located in each position, that is, the position scale indicates an angle. This manner is able to directly read the penetration path of the X-ray corresponding to each projection parameter at each relative position, thereby facilitating fast 3D reconstruction.

### Embodiment 5

This embodiment is improved on the basis of Embodiment 3, and repeated parts are not elaborated here. The position parameter extraction mechanism in this embodiment is different from the position parameter extraction mechanism in the above embodiment. The structure of the position parameter extraction mechanism in this embodiment is briefly described below.

In the present disclosure, when the motion control module uses a transmission chain matched with chain wheels or a synchronous belt matched with synchronous wheels, the position parameter extraction mechanism is able to use the solution provided in the above embodiment. Of course, identifiers may also be arranged on the driving chain wheel or the driving synchronous wheel, the position of the exposure imaging mechanism is able to be obtained according to a rotation angle of the driving chain wheel or the driving synchronous wheel, or an angle between the X-ray source and the detector is able to be directly obtained.

In this embodiment, when the motion control module uses the transmission chain matched with chain wheels, as shown in FIG. 12 and FIG. 13, the position parameter extraction mechanism includes a scale extraction camera (not shown in the figures), angle scales 3142 arranged on the driving chain wheel 3141, and a second pointer 3143 pointing to the angle scales. The driving chain wheel 3141 may be fixed on a fixing mechanism 3145 through a mounting shaft 3144. The fixing mechanism may be a detector mounting frame, or a fixing frame fixed on the ground or any device through a counterweight etc. In this embodiment, the second pointer is arranged on the fixing mechanism 3145, and the scale extraction camera is spaced apart from both the driving chain wheel and the exposure imaging mechanism. During shooting of the scale extraction camera, the X-ray source of the exposure imaging mechanism, the second pointer and the driving chain wheel with angle scales all exist in a picture, so that the angle corresponding to the projection parameter is able to be obtained according to the number of revolutions of the driving chain wheel and the angle pointed by the second pointer in a shot picture. There is no need to infer a time of X-ray emission according to a time obtained based on the projection parameter, and then find an angle corresponding to the projection parameter according to the time for shooting the picture by the scale extraction camera, thereby avoiding errors in an established 3D image caused by an error between an extracted angle parameter and an actual exposure projection operation.

After the mounting of the tracks, the motion block, the detector and the motion control module is completed, when the motion block drives the X-ray source to reach each preset relative position, the angle scale may be set according to the position indicated by the second pointer on the driving chain wheel.

In some embodiments, in order to reduce the calculation of the number of rotations, sizes of the driving chain wheel and the driven chain wheel may also be adjusted, such as increasing a size of the driving chain wheel and/or reducing a diameter of the driven chain wheel.

### Embodiment 6

This embodiment is improved on the basis of the above embodiment, and the parts in this embodiment the same as those in the above embodiment are not elaborated here.

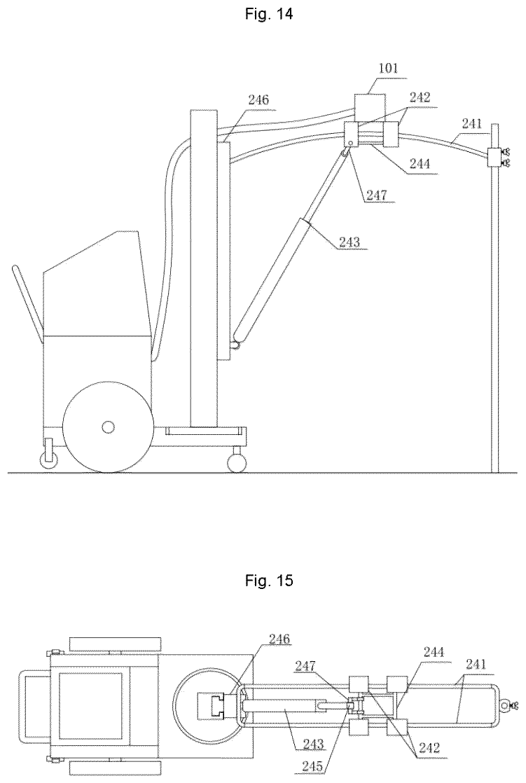

As shown in FIG. 14 and FIG. 15, in this embodiment, the two tracks are limiting sliding rods 241, and the two limiting sliding rods are arranged on the machine body at an interval side by side; the motion block includes a plurality of sliding blocks 242 sleeved on the limiting sliding rods respectively, and a third mounting frame 244 for connecting the sliding blocks together; and the X-ray source is correspondingly connected with the plurality of sliding blocks. The motion control module includes an electric telescopic rod 243 arranged below a side of the limiting sliding rod, a first end of the electric telescopic rod is fixed on the machine body, and a second end of the electric telescopic rod is connected with the third mounting frame through a bearing 245. In this embodiment, a center of a circle where the two limiting sliding rods are located is on the detector; and the two limiting sliding rods 241 are both provided with two sliding blocks 242.

In this embodiment, in order to ensure stable movement of the exposure imaging mechanism on the limiting sliding rods and also facilitate mounting, the limiting sliding rods are sleeved with the sliding blocks, each of the sliding blocks is provided with a through hole that is penetrated, the through hole is arc-shaped, and a hole diameter is slightly greater than an outer diameter of the two limiting sliding rods; and each of the two limiting sliding rods is penetrated in two through holes correspondingly, and the exposure imaging mechanism is fixed on the sliding blocks. An inner diameter of the through hole of the each of the sliding blocks is matched with the outer diameter of each of the two limiting sliding rods, so that the sliding blocks may only move along the two limiting sliding rods. Moreover, during movement, a distance between an outer wall of the each of the sliding blocks and an outer wall of the corresponding limiting sliding rod of the two limiting sliding rods remains unchanged, and then, a distance between the exposure mechanism and the each of the two limiting sliding rod remains unchanged, thereby effectively ensuring the stability of the exposure imaging mechanism during the movement along the two limiting sliding rods. In order to ensure that the distance between the exposure imaging mechanism and the object to be detected remains unchanged during the movement of the exposure imaging mechanism at each relative position, in this embodiment, the X-ray source in the exposure imaging mechanism is correspondingly connected with the motion block; and the two limiting sliding rods are arc-shaped, and a center of a circle where the arc is located is on the detector. In this embodiment, the X-ray source is welded to the sliding blocks, and the each of the sliding blocks is correspondingly connected with four corners at a bottom of the X-ray source respectively to prevent obstructing a projection angle of X-ray beams. Of course, mechanical structures such as glue or bolts may also be selected for connection. Those skilled in the art may select a connection manner according to actual needs. Details are not elaborated here.

In this embodiment, the position parameter extraction mechanism includes a mechanism for collecting a telescopic degree of the electric telescopic rod. The positions of the third mounting frame and the exposure imaging mechanism are determined through the telescopic length of the electric telescopic rod.

In this embodiment, the mechanism for collecting the telescopic degree of the electric telescopic rod consists of scales set along a length of the telescopic rod and a camera for shooting the telescopic length of the telescopic rod; and relative position parameters are determined based on the content displayed by the scales in an image shot by the camera. During setting of scales, other measurement or calculation tools may be used for calculating the distance or angle of motion of the X-ray source at each relative position, and then, the distance or angle is marked at the corresponding position on the electric telescopic rod. Of course, collection may be performed based on a structure capable of calculating the telescopic length of the telescopic rod, or an electrified time of a telescopic motor of the electric telescopic rod, the number of rotations of a motor shaft, etc. Those skilled in the art may make a selection according to needs. Details are not elaborated here.

As shown in FIG. 14 to FIG. 15, in order to further ensure the stability of motion of the sliding block controlled by the electric telescopic rod 243, the bearing 245 is fixedly connected with a free end of the electric telescopic rod, the bearing is sleeved on a U-shaped stable frame 247, a middle of the U-shaped stable frame is sleeved with the bearing 245, and both ends of the U-shaped stable frame are correspondingly penetrated on the third mounting frame through bearings respectively. In some embodiments, a distance between both ends of the U-shaped stable frame and the corresponding nearest sliding block is equal.

As shown in FIG. 14 to FIG. 15, in this embodiment, a transfer sliding block 246 is arranged in the longitudinal sliding support rod, and the transfer sliding block is able to move up and down along a length direction of the longitudinal sliding support rod; an end of the two limiting sliding rods is connected with the transfer sliding block, a fixed end of the electric telescopic rod is connected with the transfer sliding block, and a connection position between the electric telescopic rod and the transfer sliding block is located below a connection position between the two limiting sliding rods and the transfer sliding block; and the free end of the electric telescopic rod is correspondingly connected with the third mounting frame through the bearing.

In this embodiment, the U-shaped stable frame uniformly transmits a force of the electric telescopic rod to the third mounting frame, which is favorable for stably driving each of the sliding blocks on both sides of the third mounting frame to move on the two limiting sliding rods.

In this embodiment, the X-ray source is driven to move through the two limiting sliding rods, the sliding blocks and the electric telescopic rod, and the electric telescopic rod drives the sliding blocks to move on the two limiting sliding rods to successively reach each preset relative position in any order. The combination of the two limiting sliding rods and the sliding blocks has the characteristic of high control degree for the exposure imaging mechanism, which may effectively prevent the exposure imaging mechanism from jumping up and down.

In the present disclosure, the two limiting sliding rods which are arc-shaped achieve that at each relative position, the focus of the X-ray source emitting X-rays is always maintained at a fixed point on the detector without causing focus shift due to the movement of the X-ray source. Furthermore, the center of the circle where the arc is located is on the surface of the detector, and the distance between the detector and X-ray source remains unchanged when the detector or X-ray source is located at each relative position, thereby further facilitating fast 3D reconstruction.

### Embodiment 7

A 3D imaging data acquisition method provided in this embodiment includes: obtaining 3D imaging data by using the mobile 3D imaging machine based on the X-ray according to any one of the above embodiments.

The present disclosure further relates to a computer storage medium storing a computer program code. When the program code is executed, various embodiments of the method of the present disclosure may be implemented. The storage medium may be a tangible storage medium, such as an optical disk, a USB flash disk, a soft disk, or a hard disk.

Those skilled in the art should understand that the exemplary components, system and method described with reference to the implementations disclosed herein can be implemented by hardware, software, or a combination of hardware and software. Whether the contents are executed in a manner of hardware or software depends on specific applications and design constraints of the technical solutions. Those skilled in the art can implement the described functions by using different methods for each specific application, but such implementation should not be considered beyond the scope of the present disclosure. When implemented in a manner of hardware, it may be an electronic circuit, an application specific integrated circuit (ASIC), appropriate firmware, a plug-in, a functional card, or the like. When implemented in a manner of software, the elements of the present disclosure are programs or code segments used for executing the required tasks. The programs or code segments may be stored in a machine-readable medium, or transmitted on a transmission medium or communication link through a data signal carried in a carrier. The “machine-readable medium” may include any medium capable of storing or transmitting information. Examples of the machine-readable medium include an electronic circuit, a semiconductor memory device, a read-only memory (ROM), a flash memory, an erasable ROM (EROM), a soft disk, a compact disc-ROM (CD-ROM), an optical disk, a hard disk, an optical fiber medium, a radio frequency (RF) link, etc. The code segments may be downloaded through computer networks such as the Internet and the Intranet.

It should also be noted that the exemplary embodiments mentioned in the present disclosure describe some methods or systems based on a series of steps or devices. However, the present disclosure is not limited to the order of the above steps. That is to say, the steps may be executed according to the order mentioned in the embodiments or different from the order in the embodiments, or multiple steps may be executed simultaneously.

In the present disclosure, features described and/or illustrated for one implementation may be used in the same or similar manner in one or more other implementations, and/or may be combined with features of other implementations, or may replace features of other implementations.

The above descriptions are only some embodiments of the present disclosure, but are not intended to limit the present disclosure. For those skilled in the art, the embodiments of the present disclosure may have various modifications and variations. Any modifications, equivalent substitutions, improvements, and the like made within the spirit and principle of the present disclosure shall be included within the protection scope of the present disclosure.

1. A mobile three-dimensional (3D) imaging machine based on an X-ray, comprising: a machine body, and a limiting mechanism, an exposure imaging mechanism and a position parameter extraction mechanism which are mounted on the machine body, wherein
the limiting mechanism is configured to control a relative position between the exposure imaging mechanism and an object to be detected, so that the exposure imaging mechanism is able to reach different relative positions between the exposure imaging mechanism and the object to be detected in sequence;
the exposure imaging mechanism is configured to respectively collect multiple projection data of the X-ray on the object to be detected at a plurality of relative positions;
the position parameter extraction mechanism is configured to extract relative positions between the object to be detected and the exposure imaging mechanism corresponding to each of the multiple projection data, and/or relative position data between an X-ray source and a detector in the exposure imaging mechanism corresponding to each of the multiple projection data.

2. The mobile 3D imaging machine based on the X-ray as claimed in claim 1, wherein the limiting mechanism comprises at least one track in an arc shape mounted on the machine body, a motion block capable of moving along the at least one track, and a motion control module for controlling the motion block to move along the at least one track; and the X-ray source in the exposure imaging mechanism is correspondingly connected with the motion block.

3. The mobile 3D imaging machine based on the X-ray as claimed in claim 2, wherein two tracks in the at least one track are arranged on the machine body at an interval side by side, and a rack is laid in each of the two tracks along a length direction of the each of the two tracks; the motion block comprises a first mounting frame, connecting shafts penetrating through the first mounting frame, and a plurality of motion gears arranged on the connecting shafts, and each of the motion gears is respectively meshed with a rack on a corresponding track of the two tracks; and the X-ray source is correspondingly connected with the first mounting frame;
or, two tracks in the at least one track are sliding rails, and the two tracks are arranged on the machine body at an interval side by side; the motion block comprises a second mounting frame, connecting shafts penetrated through the second mounting frame, and a plurality of rollers arranged on the connecting shafts, and each of the plurality of rollers is respectively arranged in a corresponding sliding rail of the sliding rails; and the X-ray source is correspondingly connected with the second mounting frame;
or, two tracks in the at least one track are limiting sliding rods, and the two limiting sliding rods are arranged on the machine body at an interval side by side; the motion block comprises a plurality of sliding blocks sleeved on the limiting sliding rods respectively, and a third mounting frame for connecting the sliding blocks together; and the X-ray source is correspondingly connected with the plurality of sliding blocks.

4. The mobile 3D imaging machine based on the X-ray as claimed in claim 3, wherein when the motion block comprises motion gears or rollers,
the motion control module comprises a motor, a driving gear sleeved on an output shaft of the motor, and driven gears fixed on the connecting shafts, and the driving gear is correspondingly meshed with one driven gear of the driven gears;
or, the motion control module comprises a driving chain wheel, a driven chain wheel sleeved on one of the connecting shafts, a transmission chain for connecting the driving chain wheel and the driven chain wheel, and a rotating handle for driving the driving chain wheel to rotate; and
the driving chain wheel is located at a center of a circle where the two tracks are located;
or, the motion control module comprises a driving synchronous wheel, a driven synchronous wheel sleeved on one of the connecting shafts, a synchronous belt for connecting the driven synchronous wheel and the driving synchronous wheel, and a rotating handle for driving the driving synchronous wheel to rotate; and the driving synchronous wheel is located at a center of a circle where the two tracks are located.

5. The mobile 3D imaging machine based on the X-ray as claimed in claim 4, wherein when the two tracks are limiting sliding rods, the motion control module comprises an electric telescopic rod disposed below a side of the limiting sliding rods, a first end of the electric telescopic rod is fixed on the machine body, and a second end of the electric telescopic rod is connected with the third mounting frame through a bearing.

6. The mobile 3D imaging machine based on the X-ray as claimed in claim 5, wherein the position parameter extraction mechanism comprises a revolution meter arranged on the connecting shafts, and is configured to extract a current position of the X-ray source according to a number of rotations of the connecting shafts; or, the position parameter extraction mechanism comprises a plurality of optoelectronic couplers uniformly distributed along the length direction of the two tracks, and is configured to extract a current position of the X-ray source according to a position of the optoelectronic coupler that is blocked in the plurality of the optoelectronic couplers; or, the position parameter extraction mechanism comprises a scale extraction camera, position scales arranged on an outer wall of one of the two tracks along the length direction of the two tracks, and a first pointer arranged on the exposure imaging mechanism and pointing to the position scales; or, the position parameter extraction mechanism comprises a scale extraction camera, angle scales arranged on the driving chain wheel or the driving synchronous wheel, and a second pointer pointing to the angle scales.

7. The mobile 3D imaging machine based on the X-ray as claimed in claim 2, wherein the machine body comprises a mobile chassis, and an imaging control calculator, a high-voltage generator and a lifting frame which are arranged on the mobile chassis; the imaging control calculator is electrically connected with the high-voltage generator and the detector respectively; the high-voltage generator is electrically connected with the X-ray source; and the at least one track is mounted on the lifting frame.

8. The mobile 3D imaging machine based on the X-ray as claimed in claim 7, wherein the lifting frame comprises a rotating base arranged on the mobile chassis and a longitudinal sliding support rod arranged on the rotating base, and an end of the at least one track is mounted in the longitudinal sliding support rod.

9. The mobile 3D imaging machine based on the X-ray as claimed in claim 7, wherein the mobile 3D imaging machine based on the X-ray further comprises a wireless control calculator electrically connected with the imaging control calculator.

10. A 3D imaging data acquisition method, comprising: obtaining 3D imaging data by using the mobile 3D imaging machine based on the X-ray as claimed in claim 1.

11. The 3D imaging data acquisition method as claimed in claim 10, wherein the limiting mechanism comprises at least one track in an arc shape mounted on the machine body, a motion block capable of moving along the at least one track, and a motion control module for controlling the motion block to move along the at least one track; and the X-ray source in the exposure imaging mechanism is correspondingly connected with the motion block.

12. The 3D imaging data acquisition method as claimed in claim 11, wherein two tracks in the at least one track are arranged on the machine body at an interval side by side, and a rack is laid in each of the two tracks along a length direction of the each of the two tracks; the motion block comprises a first mounting frame, connecting shafts penetrating through the first mounting frame, and a plurality of motion gears arranged on the connecting shafts, and each of the motion gears is respectively meshed with a rack on a corresponding track of the two tracks; and
the X-ray source is correspondingly connected with the first mounting frame;
or, two tracks in the at least one track are sliding rails, and the two tracks are arranged on the machine body at an interval side by side; the motion block comprises a second mounting frame, connecting shafts penetrated through the second mounting frame, and a plurality of rollers arranged on the connecting shafts, and each of the plurality of rollers is respectively arranged in a corresponding sliding rail of the sliding rails; and the X-ray source is correspondingly connected with the second mounting frame;
or, two tracks in the at least one track are limiting sliding rods, and the two limiting sliding rods are arranged on the machine body at an interval side by side; the motion block comprises a plurality of sliding blocks sleeved on the limiting sliding rods respectively, and a third mounting frame for connecting the sliding blocks together; and the X-ray source is correspondingly connected with the plurality of sliding blocks.

13. The 3D imaging data acquisition method as claimed in claim 12, wherein when the motion block comprises motion gears or rollers,
the motion control module comprises a motor, a driving gear sleeved on an output shaft of the motor, and driven gears fixed on the connecting shafts, and the driving gear is correspondingly meshed with one driven gear of the driven gears;
or, the motion control module comprises a driving chain wheel, a driven chain wheel sleeved on one of the connecting shafts, a transmission chain for connecting the driving chain wheel and the driven chain wheel, and a rotating handle for driving the driving chain wheel to rotate; and
the driving chain wheel is located at a center of a circle where the two tracks are located;
or, the motion control module comprises a driving synchronous wheel, a driven synchronous wheel sleeved on one of the connecting shafts, a synchronous belt for connecting the driven synchronous wheel and the driving synchronous wheel, and a rotating handle for driving the driving synchronous wheel to rotate; and the driving synchronous wheel is located at a center of a circle where the two tracks are located.

14. The 3D imaging data acquisition method as claimed in claim 13, wherein when the two tracks are limiting sliding rods, the motion control module comprises an electric telescopic rod disposed below a side of the limiting sliding rods, a first end of the electric telescopic rod is fixed on the machine body, and a second end of the electric telescopic rod is connected with the third mounting frame through a bearing.

15. The 3D imaging data acquisition method as claimed in claim 14, wherein the position parameter extraction mechanism comprises a revolution meter arranged on the connecting shafts, and is configured to extract a current position of the X-ray source according to a number of rotations of the connecting shafts; or, the position parameter extraction mechanism comprises a plurality of optoelectronic couplers uniformly distributed along the length direction of the two tracks, and is configured to extract a current position of the X-ray source according to a position of the optoelectronic coupler that is blocked in the plurality of the optoelectronic couplers; or, the position parameter extraction mechanism comprises a scale extraction camera, position scales arranged on an outer wall of one of the two tracks along the length direction of the two tracks, and a first pointer arranged on the exposure imaging mechanism and pointing to the position scales; or, the position parameter extraction mechanism comprises a scale extraction camera, angle scales arranged on the driving chain wheel or the driving synchronous wheel, and a second pointer pointing to the angle scales.

16. The 3D imaging data acquisition method as claimed in claim 11, wherein the machine body comprises a mobile chassis, and an imaging control calculator, a high-voltage generator and a lifting frame which are arranged on the mobile chassis; the imaging control calculator is electrically connected with the high-voltage generator and the detector respectively; the high-voltage generator is electrically connected with the X-ray source; and the at least one track is mounted on the lifting frame.

17. The 3D imaging data acquisition method as claimed in claim 16, wherein the lifting frame comprises a rotating base arranged on the mobile chassis and a longitudinal sliding support rod arranged on the rotating base, and an end of the at least one track is mounted in the longitudinal sliding support rod.

18. The 3D imaging data acquisition method as claimed in claim 16, wherein the mobile 3D imaging machine based on the X-ray further comprises a wireless control calculator electrically connected with the imaging control calculator.
